(19) United States
(12) Patent Application Publication
(10) Pub. No.: US 2021/0161949 A1
(43) Pub. Date: Jun. 3, 2021

(54) PHARMACEUTICAL COMPOSITION CONTAINING DEXPANTHENOL AND POLYHEXANIDE

(71) Applicant: Bayerische Patentallianz GmbH, Muenchen (DE)

(72) Inventor: Karl BODENSCHATZ, Nuernberg (DE)

(21) Appl. No.: 17/046,445

(22) PCT Filed: Apr. 1, 2019
(86) PCT No.: PCT/EP2019/058199

§ 371 Date: Oct. 9, 2020

(30) Foreign Application Priority Data
Apr. 11, 2018 (EP) 18166795.7

(51) Int. Cl. A61K 31/785; A61P 17/02; A61K 31/16; A61K 47/10; A61K 47/34; A61K 31/573; A61K 9/00; A61K 9/06; A61K 9/107

(52) U.S. Cl. CPC A61K 31/785; A61P 17/02; A61K 31/16; A61K 47/10; A61K 9/107; A61K 31/573; A61K 9/0014; A61K 9/06; A61K 47/34

(57) ABSTRACT

The invention relates to a pharmaceutical composition containing dexpanthenol and polyhexanide for use in the topical treatment of an injury to the skin in a human or an animal, wherein the pharmaceutical composition is formulated as an emulsion with a lipophilic phase and hydrophilic phase, the hydrophilic phase containing the dexpanthenol and the polyhexanide, and the lipophilic phase containing at least one corticoid or a salt of a corticoid or an ester of a corticoid.

The invention relates to a pharmaceutical composition containing dexpanthenol and polyhexanide.

EP 1 992 340 B1 discloses an ophthalmic solution containing dexpanthenol, a phosphate buffer and polyhexamethylene biguanide (=polyhexanide). Ophthalmic solutions are to be understood as meaning solutions for treatment of contact lenses and also wetting solutions and solutions for ocular administration for treatment of eye conditions.

DE 20 2011 108 802 U1 discloses a wound dressing for therapeutic wound care, wherein the wound dressing comprises at least one wound covering layer and at least one sorbent based on an activated carbon. The activated carbon has a biocidal and/or biostatic activity and/or endowment. To this end the activated carbon may have been endowed with at least one biocidal and/or biostatic active. The active may be selected from the group of polyhexamethylene biguanide, taurolidine, benzalkonium chloride, chlorhexidine, octenidine and physiologically compatible salts and derivatives thereof and also mixtures thereof. The activated carbon may additionally have been endowed and/or impregnated with at least one further active. The further active may be an active having wound-healing promoting activity. It may be selected from the group of alginates, chitosan, hyaluronic acid and salts thereof, allantoin, beta-sitosterol, bromelain, dexpanthenol, pantothenic acid, urea, flavonoids, riboflavin, saponins, cineole, tocopherol and mixtures thereof.

EP 2 196 225 A2 discloses a preparation, in particular wound care preparations, comprising one or more film-forming hydrophobic acrylate-based polymers, water, water-soluble actives and one or more solubilizers selected from the group of isopropyl alcohol, propyl alcohol and/or 2-phenoxyethanol. The actives may be selected from the group of antiseptics, in particular benzalkonium chloride, octenidine (octenidine dihydrochloride), chlorhexidine, chlorhexidine digluconate, chlorhexidine diacetate monohydrate and/or polyhexanide, or from the group of dexpanthenol, polidocanol, witch hazel extract, magnolia extract and peony extract.

The present invention has for its object to specify an alternative pharmaceutical composition which is particularly suitable for use in the topical treatment of a skin injury in a human or an animal, in particular a mammal. The injury may be a burn injury, in particular a relatively large-area burn injury.

The object is achieved by the features of claim 1. Advantageous embodiments result from the features of claims 2 to 12.

The present invention provides a pharmaceutical composition containing dexpanthenol and polyhexanide for use in the topical treatment of a skin injury in a human or an animal, in particular a mammal. The pharmaceutical composition is formulated as an emulsion comprising a lipophilic phase and a hydrophilic phase, wherein the hydrophilic phase contains the dexpanthenol and the polyhexanide. The lipophilic phase contains at least one corticoid or a salt, in particular a physiologically acceptable salt, of a corticoid or an ester, in particular a physiologically acceptable ester, of a corticoid. The lipophilic phase may further comprise at least one constituent from a group consisting of capric acid, caprylic acid, caproic acid, lauric acid, myristic acid, paraffin, glycerol monostearate, wax, in particular bleached wax, wool wax, wool wax alcohol, almond oil, sunflower oil, castor oil, peanut oil, olive oil, mineral oil, 2-ethylhexyl laurate, decyl oleate, cetyl stearyl alcohol, in particular cetyl stearyl alcohol type A, and petrolatum, in particular white petrolatum. The hydrophilic phase may comprise at least one constituent from a group consisting of water, an alcohol, glycerol, polyethylene glycol and/or propylene glycol. Emulsifiers, for example glycerol monostearate 60, cetyl alcohol, and/or macrogol 1000 glycerol monostearate, are generally also present. The pH of the emulsion may be in the range from 4 to 7, in particular 4.5 to 6.5.

A suitable emulsion is for example so-called DAC base cream as per the German Pharmaceutical Codex [Deutscher Arzneimittel-Codex] into which dexpanthenol and polyhexanide and also a corticoid or a salt or an ester of a corticoid have been incorporated. 100 g of DAC base cream have the following composition: 4.0 g glycerol monostearate 60, 6.0 g cetyl alcohol, 7.5 g medium chain triglycerides (neutral oil, Miglyol 812), 25.5 g white petrolatum, 7.0 g macrogol 1000 glycerol monostearate, 10.0 g propylene glycol and 40.0 g purified water.

The inventor has found that the pharmaceutical composition according to the invention is particularly suitable for treatment of injuries to the skin where new skin is to be formed, in particular on a relatively large-area basis, to avoid a skin replacement operation, for example an autologous skin graft, or to reduce the required scope thereof. The pharmaceutical composition according to the invention may remain on the skin injury for several days, for example under a bandage, without any need for renewed wound care. A bandage change interval may therefore be markedly extended compared to hitherto customary intervals. The polyhexanide prevents germ-contamination of the pharmaceutical composition according to the invention as well as infection and consequent inflammatory reaction of the injured skin. The pharmaceutical composition according to the invention has even made it possible to sterilize and sanitize a skin wound contaminated with multi-resistant germs. The dexpanthenol brings about rapid skin regeneration in conjunction with the emulsion and the polyhexanide. The dexpanthenol simultaneously inhibits proliferation of fibroblasts of the connective tissue below the newly formed skin and thus the formation of excess granulation tissue and scarring. The lipophilic phase has a wound caring and wound soothing activity.

The inventor has moreover found that the treatment of a skin injury in a human or an animal with the pharmaceutical composition according to the invention results in markedly faster healing than alternating treatment with polyhexanide and dexpanthenol. This makes it possible to perform bandage changes markedly more seldom than has hitherto been customary. Especially in the case of very painful skin injuries, for example burn injuries, this also makes it possible to reduce the number of sedations often required for bandage changes. This also makes it possible to reduce the side effects associated with frequent sedations.

Particularly in the case of a lengthier healing process, especially in the case of a relatively large-area skin injury, the at least one corticoid present in the lipophilic phase has an advantageous effect on the healing process. The corticoid can inhibit or at least reduce an inflammatory reaction. An inflammatory reaction would destroy both existing and regenerated skin tissue and prevent regeneration of skin. Providing an emulsion comprising a lipophilic phase and a hydrophilic phase makes it possible to achieve simultaneous treatment of the skin injury with dexpanthenol, polyhexanide and the at least one corticoid or salt or ester thereof.

In the case of a skin injury affecting more than 10% of the body surface area, topical treatment is typically carried out initially with a pharmaceutical composition whose lipophilic phase contains no corticoid, no salt and no ester thereof. It is then possible after a number of days to switch to a treatment with the pharmaceutical composition according to the invention whose lipophilic phase contains at least one corticoid or a salt of a corticoid or an ester of a corticoid. Infected skin injuries too are normally treated with a pharmaceutical composition containing no corticoid, no salt of a corticoid and no ester of a corticoid. In the case of uninfected skin injuries affecting up to 10% of the body surface area, treatment is typically commenced with the pharmaceutical composition according to the invention whose lipophilic phase contains at least one corticoid, or a salt of a corticoid or an ester of a corticoid.

Since regions of the injury not covered by newly formed skin should typically be closed by grafting after approximately 3 to 4 weeks to prevent infection, the acceleration and improvement of the healing process achievable with the pharmaceutical composition according to the invention makes it possible for skin grafts to be completely avoided or at least reduced to a smaller area.

The corticoid may be cortisone, corticosterone, cortisol, aldosterone, deoxycorticosterone, prednisone, methylprednisolone, triamcinolone, betamethasone, paramethasone, hydrocortisone, hydrocortisone acetate, hydrocortisone 17-butyrate, triamcinolone acetonide, prednicarbate, mometasone furoate, betamethasone 17-valerate, betamethasone dipropionate, clobetasol 17-propionate, prednisolone, prednisolone 21-acetate, dexamethasone, dexamethasone 21-acetate, fluocinolone acetonide, methylprednisolone aceponate, desonide, budesonide, halcinoide, desoximethasone, clocortolone, fluticasone, fluocinoide, halcinoide, amcinonide, diflorasone diacetate, flurandrenolide or alclomethasone dipropionate.

In one embodiment of the pharmaceutical composition according to the invention the corticoid or the salt of the corticoid or the ester of the corticoid is present therein in a concentration of 0.001% to 2.5% by weight, in particular 0.005% to 2% by weight. The respective corticoid may alternatively be present in the pharmaceutical composition in the following concentrations specified for the respective corticoid: hydrocortisone 0.1% to 2% by weight, hydrocortisone acetate 0.1% to 2% by weight, hydrocortisone 17-butyrate 0.02% to 0.2% by weight, triamcinolone acetonide 0.01% to 0.2% by weight, prednicarbate 0.05% to 0.5% by weight, mometasone furoate 0.02% to 0.2% by weight, betamethasone 17-valerate 0.02% to 0.2% by weight, betamethasone dipropionate 0.01% to 0.1% by weight, clobetasol 17-propionate 0.01% to 0.1% by weight, prednisolone 0.05% to 1% by weight, prednisolone 21-acetate 0.05% to 1% by weight, dexamethasone 0.005% to 0.1% by weight, dexamethasone 21-acetate 0.005% to 0.1% by weight, fluocinolone acetonide 0.01% to 0.1% by weight, methylprednisolone aceponate 0.02% to 0.2% by weight, desonide 0.01% to 0.1% by weight, budesonide 0.005% to 0.05% by weight, halcinoide 0.01% to 0.1% by weight, desoximethasone 0.01% to 0.25% by weight, clocortolone 0.01% to 0.2% by weight, fluticasone 0.01% to 0.2% by weight, fluocinoide 0.01% to 0.1% by weight, halcinoide 0.01% to 0.1% by weight, amcinonide 0.01% to 0.1% by weight, diflorasone diacetate 0.01% to 0.1% by weight, flurandrenolide 0.01% to 0.1% by weight and alclomethasone dipropionate 0.01% to 0.2% by weight, in particular 0.04% to 0.06% by weight. In the entire text the term “% by weight” is always based on the total weight of the pharmaceutical composition according to the invention.

In clinical experiments it has proven advantageous when dexpanthenol is present in the pharmaceutical composition according to the invention in a concentration of 1% to 10% by weight, in particular 1.5% to 5.5% by weight, and/or polyhexanide is present in the pharmaceutical composition according to the invention in a concentration of 0.005% to 0.3% by weight, in particular 0.01% to 0.2% by weight.

The pharmaceutical composition according to the invention may be formulated as a cream or lotion. To treat the injury the cream or lotion may be applied directly to the injured site on the skin. It is also possible to coat a wound dressing with the cream or impregnate it with the lotion and then apply said dressing to the injured site on the skin and optionally secure it with a bandage.

When the pharmaceutical composition is formulated as a cream it may contain glycerol monostearate 60, cetyl alcohol, at least one triglyceride, in particular a medium chain triglyceride, petrolatum, especially white petrolatum, macrogol 1000 glycerol monostearate, propylene glycol and water. Medium chain triglycerides are triglycerides whose fatty acids have an aliphatic chain of 6 to 12 carbon atoms, for example caproic acid (C₆H₁₂O₂), caprylic acid (C₈H₁₆O₂), capric acid (C₁₀H₂₀O₂) or lauric acid (C₁₂H₂₄O₂). A mixture suitable here as medium chain triglycerides may consist of capric acid and caprylic acid. Such a mixture is known as neutral oil and is marketed under the name Miglyol 812 for example. Glycerol monostearate 60, cetyl alcohol and macrogol 1000 glycerol monostearate are used as emulsifiers. The basis for the recited cream may be the abovementioned DAC base cream.

The injury to the skin may be a burn injury. In burn injuries, especially burn injuries in children, the pharmaceutical composition according to the invention has proven particularly suitable for bringing about relatively rapid regeneration of skin. The burn injury may be a second or third degree burn.

Treatment may be carried out at least once, twice or three times per week, in particular at least once per day, in particular twice per day, as required. However, the long treatment intervals achievable with the pharmaceutical composition according to the invention also make it possible to carry out the treatment at most once, twice or three times per week.

The invention is hereinbelow more particularly elucidated with reference to an embodiment.

A pharmaceutical composition according to the invention was produced according to the following formulation:
hydrocortisone acetate: 2.0 g
20% polyhexanide solution: 0.8 g
dexpanthenol: 20 g
DAC base cream: up to 400.0 g

The composition was produced by initially charging 50 g of the DAC base cream into a mortar and weighing in the actives according to the above table. The mixture was then stirred with a pestle at room temperature for 3 min until a homogeneous basis was formed. The mixture was then made up to 400 g with DAC base cream and stirred with a pestle for 3 min until a homogeneous cream was formed. The resulting cream contained 5 mg/g of hydrocortisone acetate, 0.4 mg/g of polyhexanide and 50 mg/g of dexpanthenol.

A further pharmaceutical composition was produced according to the following formulation:
20% polyhexanide solution: 0.8 g
dexpanthenol: 20 g
DAC base cream: up to 400.0 g

The composition was produced by initially charging 50 g of the DAC base cream into a mortar and weighing in the actives according to the above table. The mixture was then stirred with a pestle at room temperature for 3 min until a homogeneous basis was formed. The mixture was then made up to 400 g with DAC base cream and stirred with a pestle for 3 min until a homogeneous cream was formed. The resulting cream contained 0.4 mg/g of polyhexanide and 50 mg/g of dexpanthenol.

The patient treated with the above pharmaceutical compositions was a two-year-old child having second and third degree scalding by water on the thorax, abdomen, shoulder, upper and lower arm and circumferentially on the wrist and dorsally on the right hand and on the right middle finger. Altogether about 7% of body surface area was affected. Three days after the scalding the large-area scalds were covered with a synthetic skin substitute material (Suprathel® from PolyMedics Innovations GmbH, 73770 Denkendorf, Germany). After two days the skin substitute material was removed and the scalds treated with the pharmaceutical composition according to the invention twice per day by application to the affected areas. Seven days later the treatment was switched to the further pharmaceutical composition by applying the further pharmaceutical composition twice a day on the affected areas. After three days the affected areas were then treated only with a pharmaceutical composition containing dexpanthenol until completely healed. Treatment with the pharmaceutical composition according to the invention made it possible to achieve a relatively short time to healing, relatively little scarring and good healing of the affected areas.

1. A pharmaceutical composition containing dexpanthenol and polyhexanide for use in the topical treatment of a skin injury in a human or an animal, wherein the pharmaceutical composition is formulated as an emulsion comprising a lipophilic phase and a hydrophilic phase, wherein the hydrophilic phase contains the dexpanthenol and the polyhexanide, wherein the lipophilic phase contains at least one corticoid or a salt of a corticoid or an ester of a corticoid.

2. The pharmaceutical composition as claimed in claim 1 for use as in claim 1, wherein the corticoid is cortisone, corticosterone, cortisol, aldosterone, deoxycorticosterone, prednisone, methylprednisolone, triamcinolone, betamethasone, paramethasone, hydrocortisone, hydrocortisone acetate, hydrocortisone 17-butyrate, triamcinolone acetonide, prednicarbate, mometasone furoate, betamethasone 17-valerate, betamethasone dipropionate, clobetasol 17-propionate, prednisolone, prednisolone 21-acetate, dexamethasone, dexamethasone 21-acetate, fluocinolone acetonide, methylprednisolone aceponate, desonide, budesonide, halcinoide, desoximethasone, clocortolone, fluticasone, fluocinoide, halcinoide, amcinonide, diflorasone diacetate, flurandrenolide or alclomethasone dipropionate.

3. The pharmaceutical composition as claimed in claim 1 for use as in claim 1, wherein the corticoid or the salt of the corticoid or the ester of the corticoid is present therein in a concentration of 0.001% to 2.5% by weight, in particular 0.005% to 2% by weight, or the respective corticoid is present therein in the following concentrations specified for the respective corticoid: hydrocortisone 0.1% to 2% by weight, hydrocortisone acetate 0.1% to 2% by weight, hydrocortisone 17-butyrate 0.02% to 0.2% by weight, triamcinolone acetonide 0.01% to 0.2% by weight, prednicarbate 0.05% to 0.5% by weight, mometasone furoate 0.02% to 0.2% by weight, betamethasone 17-valerate 0.02% to 0.2% by weight, betamethasone dipropionate 0.01% to 0.1% by weight, clobetasol 17-propionate 0.01% to 0.1% by weight, prednisolone 0.05% to 1% by weight, prednisolone 21-acetate 0.05% to 1% by weight, dexamethasone 0.005% to 0.1% by weight, dexamethasone 21-acetate 0.005% to 0.1% by weight, fluocinolone acetonide 0.01% to 0.1% by weight, methylprednisolone aceponate 0.02% to 0.2% by weight, desonide 0.01% to 0.1% by weight, budesonide 0.005% to 0.05% by weight, halcinoide 0.01% to 0.1% by weight, desoximethasone 0.01% to 0.25% by weight, clocortolone 0.01% to 0.2% by weight, fluticasone 0.01% to 0.2% by weight, fluocinoide 0.01% to 0.1% by weight, halcinoide 0.01% to 0.1% by weight, amcinonide 0.01% to 0.1% by weight, diflorasone diacetate 0.01% to 0.1% by weight, flurandrenolide 0.01% to 0.1% by weight and alclomethasone dipropionate 0.01% to 0.2% by weight.

4. The pharmaceutical composition as claimed in claim 1 for use as in claim 1, wherein the lipophilic phase comprises at least one constituent from a group consisting of capric acid, caprylic acid, caproic acid, lauric acid, myristic acid, paraffin, glycerol monostearate, wax, wool wax, wool wax alcohol, almond oil, sunflower oil, castor oil, peanut oil, olive oil, mineral oil, 2-ethylhexyl laurate, decyl oleate, cetyl stearyl alcohol and petrolatum.

5. The pharmaceutical composition as claimed in claim 1 for use as in claim 1, wherein the hydrophilic phase comprises at least one constituent from a group consisting of water, an alcohol, glycerol, polyethylene glycol and propylene glycol.

6. The pharmaceutical composition as claimed in claim 1 for use as in claim 1, wherein dexpanthenol is present therein in a concentration of 1% to 10% by weight, in particular 1.5% to 5.5% by weight, and/or polyhexanide is present therein in a concentration of 0.005% to 0.3% by weight, in particular 0.01% to 0.2% by weight.

7. The pharmaceutical composition as claimed in claim 1 for use as in claim 1, wherein said composition is formulated as a cream or lotion.

8. The pharmaceutical composition as claimed in claim 1 for use as in claim 1, wherein said composition is formulated as a cream containing glycerol monostearate 60, cetyl alcohol, at least one triglyceride, petrolatum, macrogol 1000 glycerol monostearate, propylene glycol and water.

9. The pharmaceutical composition as claimed in claim 1 for use as in claim 1, wherein the skin injury is a burn injury.

10. The pharmaceutical composition as claimed in claim 1 for use as in claim 1, wherein the burn injury is a second or third degree burn.

11. The pharmaceutical composition as claimed in claim 1 for use as in claim 1, wherein the treatment is carried out at least once, twice or three times per week, in particular at least once per day.

12. The pharmaceutical composition as claimed in claim 1 for use as in claim 1, wherein the treatment is carried out at most once, twice or three times per week.
